(19) United States
(12) Patent Application Publication
(10) Pub. No.: US 2010/0158873 A1
(43) Pub. Date: Jun. 24, 2010

(54) METHOD FOR EXTRACTING AND SELECTING CELLS

(76) Inventor: Christian Pinset, Paris (FR)

Correspondence Address: ADELI & TOLLEN, LLP, 11940 San Vicente Blvd., Suite 100, LOS ANGELES, CA, 90049, US

(21) Appl. No.: 12/441,382

(22) PCT Filed: Sep. 14, 2007
(86) PCT No.: PCT/FR2007/001497

§ 371 Date: Jan. 26, 2010

(30) Foreign Application Priority Data
Sep. 15, 2006 (EP) 06291462.7

(51) Int. Cl. A61K 35/34; A61K 35/12; C12Q 1/02; C12N 5/077; C12N 5/02

(52) U.S. Cl. 424/93.7; 435/29; 435/325; 435/374; 435/408

(57) ABSTRACT

Method for selecting cells, comprising at least one at least partially combined step of enzymatic digestion and of selection in culture.

### FIELD OF THE INVENTION

The present invention relates to methods for extracting, selecting, and preserving cells for the production of cells that can be used for cell therapy and pharmacology as well as specifically adapted culture media.

### PRIOR ART

The work by Cosimo de Bari et al. (Arthritis and Rheumatism, Vol. 44, No. 8, August 2001, p. 1928 to 1942) describes obtaining synovial cells by the digestion of synovial membrane with collagenase for 24 hours in culture.

Document WO2004/055174 describes a method for selecting and then amplifying myoblasts. The selection step follows the step of extraction of the muscle cells by enzymatic digestion.

### SUMMARY OF THE INVENTION

The invention relates to a method for selecting cells, preferably muscle cells, combining, in the same step or at least partially, enzymatic extraction and selection.

The invention also relates to the cells thus obtained and to their therapeutic use. The invention also relates to a method for selecting cells, preferably muscle cells, by freezing in a medium containing trehalose. The invention also relates to culture media specifically suited to the application of these methods.

More specifically the invention relates to:
1. A method for selecting cells comprising at least one at least partially combined step of enzymatic digestion and selection in culture.
2. A method according to point 1 in which the source of the cells is a tissue sample and preferably a muscle biopsy.
3. A method according to point 1 in which the source of the cells is a cell culture and preferably an aggregate of cells in culture.
4. A method according to points 1 to 3 in which the cells selected are muscle cells.
5. A method according to points 1 to 4 in which the cells selected are skeletal muscle stem cells, muscle stem cells, muscle precursor stem cells, myoblasts or satellite cells.
6. A method according to points 1 to 5 in which selection is carried out at least by culture in a selection medium.
7. A method according to points 1 to 5 in which selection is carried out at least by adhesion to a culture substrate.
8. A method according to points 1 to 5 in which selection is carried out at least by adhesion to a substrate and by culture in a selection medium.
9. A method according to points 7 to 8 in which the substrate is chosen from the group comprising glass, treated plastic, synthetic substrates, nutrient cells, antibodies, peptides and constituents of the extracellular matrix, preferably laminin, fibronectin, vitronectin.
10. A method according to point 9 in which the adherent cells are selected.
11. A method according to point 9 in which the non-adherent cells are selected.
12. A method according to one of the previous points in which selection is carried out by culture in a selection medium comprising at least dexamethasone, selenium, and one or more compounds chosen from the group comprising ascorbic acid 2-phosphate, ascorbic acid and mixtures thereof
13. A method according to one of the previous points in which the culture medium includes the enzyme used for enzymatic digestion.
14. A method according to one of the previous points in which the selection medium includes the enzyme used for enzymatic digestion.
15. A method according to one of the previous points in which the enzymatic extraction uses an enzyme selected from the group comprising collagenase, neutral protease, pronase, trypsin and mixtures thereof
16. A method according to one of the previous points in which the combined step of enzymatic extraction and selection takes at least 3 hours, preferably at least 6 hours, more preferably at least 12 hours, even more preferably at least 24 hours, and most preferably at least 48 hours.
17. A method for selecting muscle cells by preservation in the presence of a compound chosen from the group comprising sugars, trehalose, glycine, HES (hydroxyethyl starch), glycerol and arbutin.
18. A method according to point 17 in which the source of the cells is a tissue sample and preferably a muscle biopsy.
19. A method according to point 17 in which the source of the cells is a cell culture and preferably an aggregate of cells in culture.
20. A method according to points 17 to 19 in which the cells selected are skeletal muscle stem cells, muscle stem cells, muscle precursor stem cells, myoblasts or satellite cells.
21. A method according to points 17 to 20 in which preservation comprises at least one freezing step.
22. A method according to the previous point in which freezing is carried out in the presence of trehalose.
23. A method according to one of points 21 or 22 in which the concentration of trehalose is between 1 mM and 1 M, preferably 0.2 M.
24. Cells obtainable by the methods for selecting cells in the previous points.
25. Muscle cells obtainable by the methods for selecting cells in the previous points.
26. Muscle cells obtainable by the methods for selecting cells according to one of the previous points for use thereof in cell therapy.
27. Muscle cells according to point 26 for use thereof in the functional treatment of the muscles.
28. Muscle cells according to the previous point for use thereof in the functional treatment of the small muscles.
29. Muscle cells according to the previous point for use thereof in the functional treatment of the sphincters.
30. Muscle cells according to the previous point for use thereof in the functional treatment of urinary incontinence.
31. Muscle cells according to point 29 for use thereof in the functional treatment of anal incontinence.
32. A culture medium substantially insulin-free comprising at least dexamethasone, selenium, and one or more compounds chosen from the group comprising ascorbic acid 2-phosphate, ascorbic acid and mixtures thereof
33. A culture medium comprising at least dexamethasone, selenium, ascorbic acid 2-phosphate and ascorbic acid.
34. A culture medium according to points 32 or 33 additionally comprising serum, preferably fetal calf serum or human serum.
35. A culture medium according to the previous point in which the serum concentration by volume is at least 5% and preferably 10%.
36. A culture medium according to the previous point comprising at least dexamethasone at a concentration of approximately 5.10⁻⁹ M, ascorbic acid 2-phosphate at a concentration of approximately 1 mM, ascorbic acid at a concentration of 0.252 mM and sodium selenite at a concentration of 250 nM.
37. A culture medium according to points 32 to 36 additionally comprising an enzyme for enzymatic digestion.
38. A culture medium according to the previous point in which the enzyme is chosen from the group comprising collagenase, neutral protease, pronase, and trypsin.
39. A cell preservation medium comprising trehalose and DMSO.
40. A cell preservation medium according to the previous point in which the final concentration of trehalose is between 1 mM and 1 M, preferably 0.2 M.
41. A cell preservation medium according to one of points 39 to 40 additionally comprising serum.
42. A cell preservation medium according to the previous point comprising from 0 to 90% serum by volume, from 0 to 20% DMSO by volume and from 1 mM to 1 M of trehalose.
43. A cell preservation medium according to one of points 39 to 40 additionally comprising albumin.
44. A cell preservation medium according to the previous point comprising from 1 to 50% of albumin by volume, from 0 to 20% of DMSO by volume and from 1 mM to 1 M of trehalose.

Generally, culture consists of placing cells in a medium, under conditions suitable for maintaining cell life for a prolonged period of time.

Generally, a method for selecting cells comprising a combined step of enzymatic digestion and of selection in culture is implemented by using a step of selection in culture, during which enzymatic digestion is carried out. More specifically, during this culture phase, selection is carried out by the medium (and/or by adhesion to a substrate) at the same time as digestion. This combined step can take at least 3 hours, preferably at least 6 hours, more preferably at least 12 hours and even more preferably at least 24 hours, and most preferably at least 48 hours. Generally, the selection in culture results in the survival or the selective development of certain cells or of certain cell types. At the end of this step certain cells or certain cell types will have been selected.

Generally, the selection of muscle cells by preservation is implemented by the survival and/or the selective development of certain cell types following a preservation step. Generally, preservation consists of maintaining survival and cell viability by the suspension or the slowing of cellular metabolism. Generally, preservation is carried out by cooling or freezing. Cooling or freezing results in the survival and/or the selective development of certain cells or of certain cell types. At the end of this step certain cells or certain cell types will have been selected. This selection depends on both the preservation time and temperature.

Generally, a substantially insulin-free culture medium is utilized by using a culture medium that does not contain insulin or contains only the insulin contained in the serum used in this medium. The concentration of insulin in such a medium does not exceed the concentration of insulin in the serum. In contrast, a medium supplemented with insulin would typically contain an amount of insulin a thousand times greater than that present in the serum.

The figures are described below:

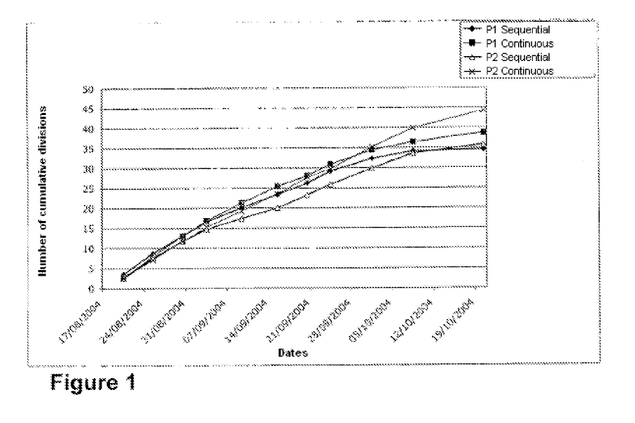

FIG. 1 shows the cumulative division number as a function of time for cells obtained from patient 1 and selected by sequential extraction (represented by diamonds on the graph), or selected by continuous extraction (represented by squares on the graph) as well as cells obtained from patient 2 and selected by sequential extraction (represented by triangles on the graph), or selected by continuous extraction (represented by crosses on the graph).

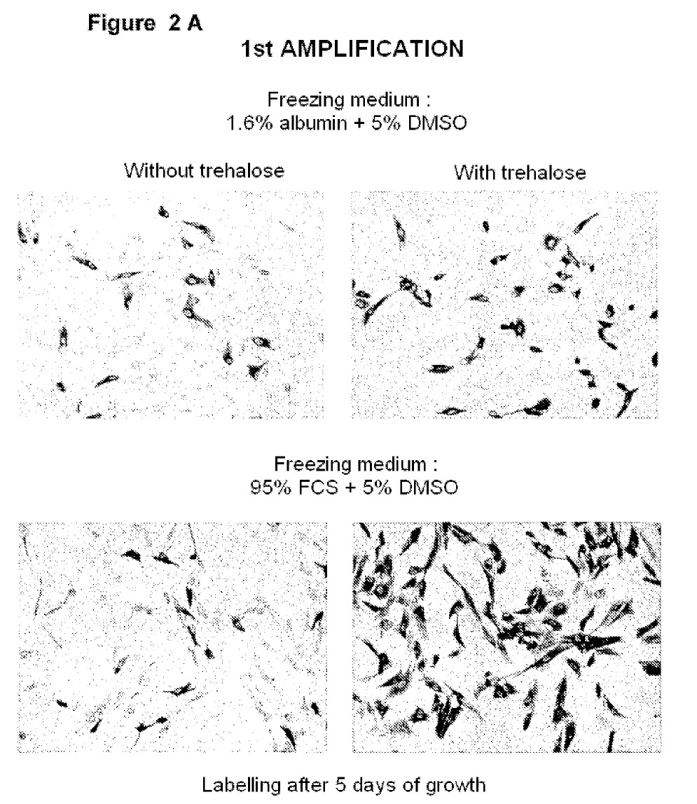

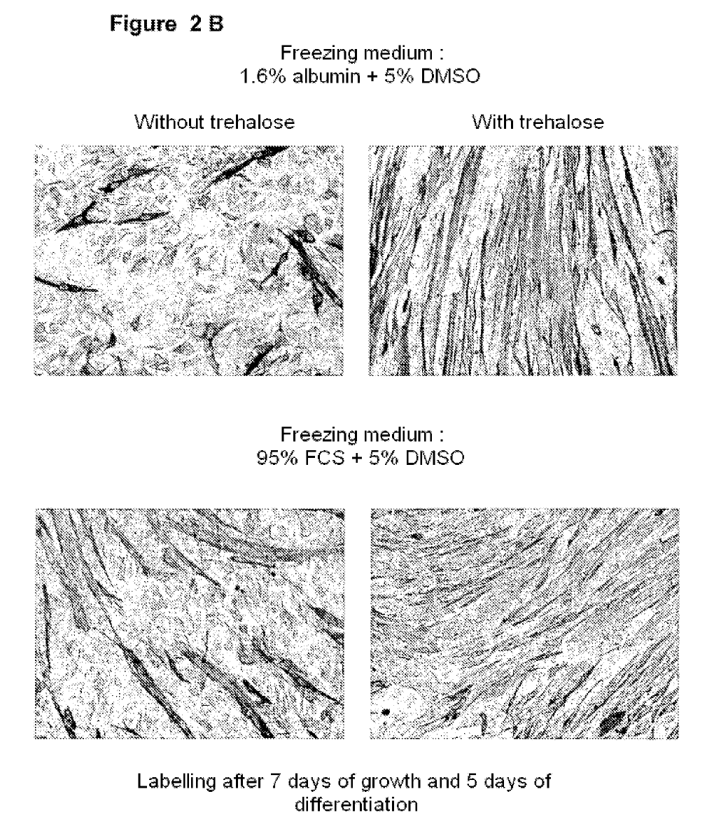

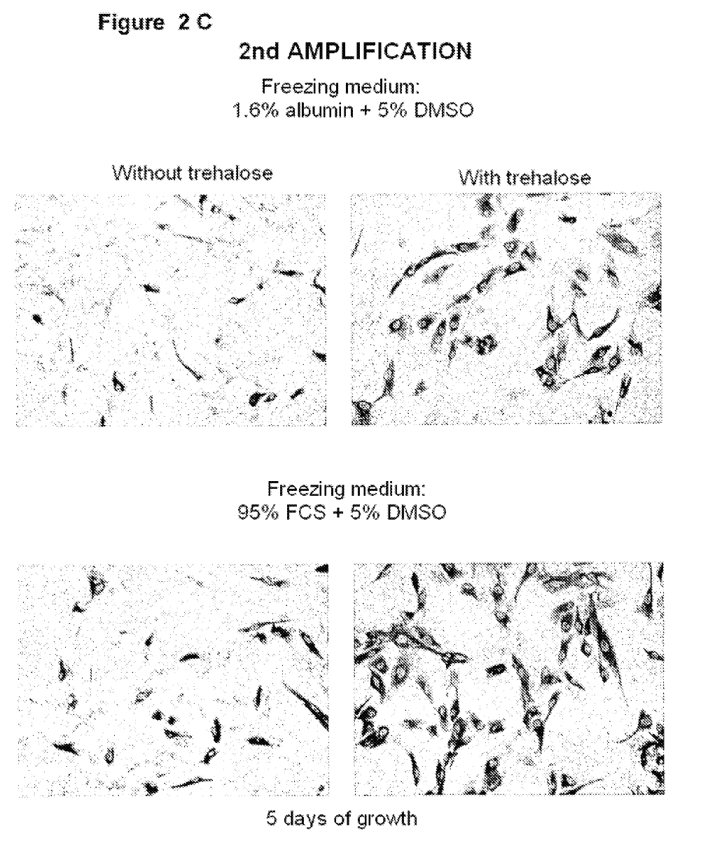

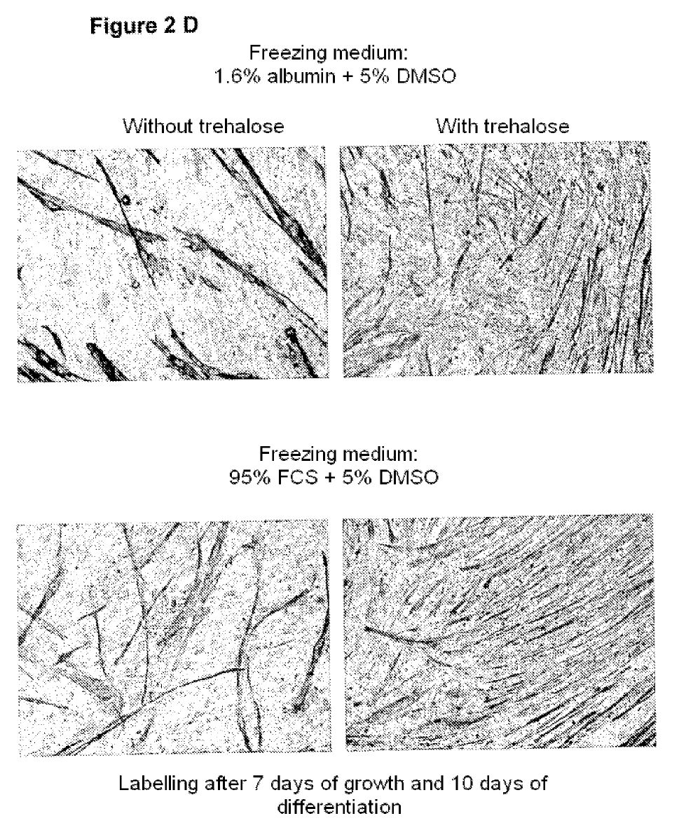

FIG. 2 shows cells resulting from extraction and selection methods on muscle tissue that had previously been preserved by freezing in the absence or in the presence of trehalose. The muscle cells appear stained brown.

FIG. 2A compares the labelling for desmin during a first amplification after 5 days of growth for two media, one with 1.6% albumin+5% DMSO (figures at the top) and the other with 95% FCS+5% DMSO (figures at the bottom), in both cases with or without addition of trehalose at a final concentration of 0.2M (the left-hand column represents the results without trehalose and the right-hand column, those with trehalose). FIG. 2B compares the labelling for desmin during a first amplification after 7 days of growth and 5 days of differentiation for two media, one with 1.6% albumin+5% DMSO (figures at the top) and the other with 95% FCS+5% DMSO (figures at the bottom), in both cases with or without addition of trehalose at a final concentration of 0.2 M (the left-hand column represents the results without trehalose and the right-hand column those with trehalose). FIG. 2C compares the labelling for desmin during a second amplification after 5 days of growth for two media, one with 1.6% albumin+5% DMSO (figures at the top) and the other with 95% FCS+5% DMSO (figures at the bottom), in both cases with or without addition of trehalose at a final concentration of 0.2M (the left-hand column represents the results without trehalose and the right-hand column those with trehalose). FIG. 2D compares the labelling for desmin during a second amplification after 7 days of growth and 10 days of differentiation for two media, one with 1.6% albumin+5% DMSO (figures at the top) and the other with 95% FCS+5% DMSO (figures at the bottom), in both cases with or without addition of trehalose at a final concentration of 0.2M (the left-hand column represents the results without trehalose and the right-hand column those with trehalose).

### DESCRIPTION OF THE INVENTION

The present invention relates to a method for producing and preserving cells, preferably muscle cells, from biological tissues or from cell aggregates. One of the aims of this novel method is to simplify and to increase the effectiveness of the various procedures required for obtaining and preserving cells for cell therapy and for pharmacology and for the constitution of a biological tissue bank from tissue biopsy. These simplifications of the production procedures facilitate the automation of the culture methods and ensure the safety of the production methods.

In the cell extraction and selection methods of the prior art, it was first necessary to have a step of enzymatic extraction, then, after washing and neutralization of the enzymes, a separate step for selecting the cells. The enzymatic extraction was of short duration and was separate from the steps of seeding and selection.

It was discovered that it is possible to combine, at least partially, the step of extraction of cells from tissues (or from cell aggregates) and the step of cell selection by culture. Moreover, this combination leads, particularly surprisingly, to a quantitative improvement (extraction yield) and qualitative improvement (the potential for growth and for differentiation). This method is simpler, quicker to implement and more effective for the production of cells for therapeutic use.

The selection step can be carried out on the basis of different criteria, such as the ability to respond to specific growth factors leading to cell survival, different cell growth or different phenotypic development. Another criterion is the selective adhesion ability. These criteria can be combined by culture in a selected medium and on a substrate allowing selective adhesion.

This extraction method combined with the continuous selection step in particular makes it possible to:
carry out extraction by enzymatic digestion under conditions allowing the viability and the biological properties of the cells to be maintained;
combine extraction and cell selection in a single step;
carry out this extraction and selection method on directly frozen tissues;
reduce the number of human manipulations;
reduce the variability of the production method;
reduce the culture time for production of the number of cells required for therapeutic applications;
produce cells having the specifications of therapeutic cells for cell therapy both with respect to safety and effectiveness.

The source of the cells can be a biopsy or alternatively a cellular aggregate obtained from primary or secondary cell culture. The combined methods of selection and digestion of the invention can also be used for all cells that can be extracted from a group of cells. The invention can therefore be applied both to tissues obtained from a biopsy and to cell cultures in particular containing adherent cells or aggregates of cells.

The different steps of the method are described below.

### 1) Cell Extraction and Selection

The principle of this extraction method is to initiate enzymatic cell extraction directly in the culture media allowing 1) maintenance of the biological properties of the cells, including survival, 2) differential attachment and 3) cell proliferation.

For this purpose, it is necessary to select enzymes the action of which is not inhibited by growth factors or serum components, and which do not alter the cell potential. Another element of choice is to be able to use enzymes that do not present a potential health risk. The culture media are composed of medium of type DME/F12 aMEM, MCDB 120 and growth factors as well as serum components of animal or human origin. The types of enzymes used are collagenase, liberase, neutral protease, pronase, separately or in combinations. It is possible for chelating agents of the EDTA or EGTA type to be added to these.

The various elements of the cell extraction method are:

### Mechanical Dissociation

The biopsy fragments are cut into small pieces mechanically using surgical scissors or disposable scalpels. This step is optional. After this step of mechanical dissociation, the tissue fragments can be frozen directly under suitable conditions. The freezing media are either the media already defined or media containing animal or human sera. The cryopreserving agent used can be glycerol, DMSO, trehalose, sugars in the form of monosaccharides or polysaccharides, glycine, and HES (hydroxyethyl starch). This preservation can constitute a selection step.

### Enzymatic Extraction

The comminuted fragments are taken up in the culture media in the presence of enzymes and are placed in culture bottles. These culture bottles are incubated for periods of up to several days in an incubator at 37° in the presence of gases including CO₂ and oxygen. The digestion time can exceed 48 hours.

This step makes it possible to release the cells from the tissue under conditions maintaining their viability, their preferential adhesion ability and their ability to respond to growth factors.

This method also makes it possible to simplify the procedures by limiting the number of technical manipulations which, in this method, are reduced to bringing the tissue fragments in contact with the enzyme solution under conditions suitable for cell culture. This step can therefore be easily automated. The concentrations of enzymes used are variable. In this stage, the culture media are composed of media of the type DME/F12 aMEM, MCDB 120 and growth factors and serum components of animal or human origin. The choices of culture media make it possible to maintain cell viability during the period of extraction and to initiate growth and selection. As an example, it is possible to use culture media of the type DME/F12 aMEM, MCDB 120 with the addition of animal or human proteins. For this stage it is also possible to use completely synthetic media. Said proteins can be of extractive or synthetic origin. Among these proteins, it is possible to use serum proteins (serum, albumin or transferrin) and growth factors (FGFs, HGF, IGFs, VEGF, EGF or heregulin). It is also possible to add vitamins (vitamin C, derivatives of vitamin A, vitamin B or vitamin D), hormones (steroids, insulin, hormones or thyroid hormones) and pharmacological agents (agonists, antagonists, inhibitors or activators of metabolic pathways) to the culture media.

These extraction methods can be carried out on fresh tissue fragments or on frozen tissue fragments. This method makes it possible to prolong the period of enzymatic treatment without altering the cell viability and initiating selection stages. Cell selection

Cellular tissues are always composed of numerous cell types and one of the aims of this method is to increase the homogeneity and the functionality of the cells produced using this extraction and selection method. There are numerous strategies for achieving these aims. During the stage of enzymatic dissociation, cell selection can be based on biological properties (survival ability, selective cytotoxicity, gene expression, adhesion ability). In this method, selection is preferably based on at least two biological properties.

### Definition of the Selection Media

Cell survival depends both on the type of cells and on combinations of elements such as sources of carbon (glucose, galactose, fructose or pyruvate), amino acids, sources of gases (O₂ or CO₂), sources of growth factors, sources of vitamins and sources of purine and pyrimidines.

The definition of the type of selection media will depend on the type of cells to be obtained. The ability to metabolize different sources of carbon, to respond to growth factors, and to exhibit dependence on certain vitamins is variable depending on the cell types.

The methods described are particularly suitable for the selection of:

1) skeletal muscle stem cells. This cell population is derived from a skeletal muscle biopsy which possesses the potential for cell replication and the potential for differentiation into several distinct lineages including at least the skeletal muscle. At this stage, this population does not express desmin.
2) Muscle precursor stem cells. This cell population is derived from a skeletal muscle biopsy which possesses the potential for cell replication and the potential for differentiation into the lineage of the skeletal muscle. At this stage this population does not express desmin.
3) Myoblasts. This cell population is derived from a skeletal muscle biopsy which possesses the potential for cell replication, and a proportion of which expresses desmin.

These three types of cells thus defined are called muscle cells. The muscle cells produced by these methods are particularly useful for autologous or allogenic cell therapy.

Document WO2004/055174 describes selection media suitable for the production of myoblasts. The invention also describes improved selection media. These media can be insulin-free and/or contain ascorbic acid 2-phosphate, which is a precursor of ascorbic acid and makes it possible to increase the bioavailability and the stability in vitro of ascorbic acid for cell culture.

### Use of Substrates for Selection by Differential Adhesion

During the step of enzymatic dissociation, the tissue fragments can be placed on culture supports in the presence of various types of substrates. Using this method, it is therefore possible to separate the cells that will adhere to the substrate (adherent cells) and the cells that will not adhere to the substrate (non-adherent cells).

Various types of substrates can be used for this purpose:

Synthetic substrates such as glass, untreated plastics (bacteriology dish), plastics treated with synthetic substrates such as polyornithine and poly-L-lysine.
Biological substrates such as proteins of the extracellular matrix (laminin, fibronectin, or vitronectin), the reconstituted extracellular matrix, “feeders” of nutrient cells or plates covered with specific antibodies or specific peptides.

After a determined time, it is possible to separate two types of cells: non-adherent cells and adherent cells.

The non-adherent cells are recovered using a pipette. This cell suspension of non-adherent cells thus obtained is a source of cells for cell preservation, cell amplification and cell therapy.

The adherent cells are the cells that remain attached to the substrates after pipetting. The cells are then either amplified directly or subcultured using trypsin EDTA solution. These cells are also a source of cells for cellular storage, cell amplification and cell therapy.

### 2) Cell Amplification

After the selection phase, it may be useful to proceed to an amplification phase, during which the number of cells increases by successive cell division in a growth medium. Culture media suitable for cell amplification are known to a person skilled in the art and include, for the myoblasts, those disclosed in patent application WO2004/055174 in addition to those disclosed by the present invention.

### 3) Cell Preservation

Finally a freezing step can be implemented. This allows the long-term storage of the cells produced, for later use. Document WO2004/055174 describes methods of freezing which are suitable for muscle cells. The tissues and the cells can be frozen in media that are completely free from animal proteins and in the presence of cryo-protective agents such as trehalose, which allows the selective preservation of muscle cells.

### 4) Functional Cell Characterization

After the stage of selection, amplification or before use of the cells, characterization of these cells may be required. We define functional and phenotypic characterizations, which are essential for using cells as therapeutic cells for repairing a particular type of cell.

### Cell Growth Test

Systematic evaluation of the doubling time makes it possible to characterize the kinetics of cell growth of each sample produced.

This test makes it possible to define the cell coverage rate and the doubling time, reflecting the intrinsic cell activity. If the doubling time is too great (>35 hours), the reinjected cells have potentially lost their regenerative ability in vivo.

A defined quantity of cells is seeded in multiwell plates. Following deposition of cell growth medium, quantification of the cellular contents obtained between 6 hours and 24 hours makes it possible to define the cell coverage rate. Successive readings at defined intervals of culture time allow the cell doubling time to be measured.

Once fixed, the plates can be analyzed by an automated device or visually by an operator.

This technology can be controlled by programmable software in order to obtain automatic quantification of the cells based on microscopic analysis.

The automatic analysis is carried out in 3 stages, namely image acquisition, conversion of the images to digital codes by acquisition software and the optional representation of the digital codes in the form of graphs for validation, by means of operational software.

Quantitative image analysis using software suitable for the inverted optical microscope Eclipse TE2000™ makes it possible to define the number of cells obtained, and therefore their growth ability. In fact, programming the software makes it possible, starting from the quantity of cells, to calculate the number of divisions obtained and to deduce the doubling time from the number of divisions.

### Clonogenic Test

This test makes it possible to monitor the samples by investigating the cells' ability to form colonies (clonal efficiency) and to undergo differentiation.

The principle is based firstly on analysis of growth at low density in a growth (or expansion) medium and secondly on evaluation of the ability to differentiate in a specific differentiating medium. The cells are seeded in a growth medium for 14 days. Half the cells are fixed and stained after 14 days and the other half are fixed and stained after 7 additional days of culture in a differentiating medium.

This is followed by: 1) macroscopic observation, which makes it possible to count the total number of colonies and thus determine the percentage of cells displaying the potential for clonal growth, called clonal efficiency; and 2) microscopic observation, which makes it possible to determine the number and percentage of colonies of muscle cells. For the muscle cells, the muscle precursor colonies form spindle-shaped polynucleated cells by cell fusion: the myotubes, which will form the muscle fibres in the organism.

A colony is considered to be composed of muscle precursors when it contains at least one myotube, knowing that the myotube contains at least 3 nuclei. This percentage represents the rate of differentiation.

Finally, samples containing 50 cells are seeded in bottles of 25 cm², the counted number of colonies corresponding to the direct percentage of cells having clonal ability. The number of cells that have differentiated to myogenic colonies in the bottles containing differentiating medium is also observed. This test therefore makes it possible to define the clonal efficiency and differentiation rate.

### Phenotypic Characterization

This characterization is based on the detection of a phenotypic marker. As an example, for muscle it is possible to use a muscle structure protein, the desmin intermediate filament. The myoblasts are desmin-positive. Specific labelling of this muscle protein by means of a monoclonal antibody allows identification and measurement of the cell population studied. This analysis consists of labelling the cells fixed after culture with a primary antibody specific to human desmin; then this antibody is recognized by a secondary antibody revealed by a DAB-stained peroxidase. This labelling is visualized in white light. This visualization could also be achieved with fluorescent markers. This type of labelling also offers the advantage of being stable over time and resistant to light.

The analysis principle is based on 1) determination of the total number of cells by Giemsa staining, 2) determination of the proportion of desmin-positive labelled cells by labelling with a specific primary antibody and 3) optionally the development of an automatic counting system in order to eliminate the variations due to visual reading of the plates.

For this purpose, an equivalent number of cells is deposited successively in the wells of 12-well plates. 4 wells are stained with Giemsa stain, 4 other wells are tested without primary antibody and 4 are labelled with the desmin-specific antibody.

The proportion of desmin-labelled therapeutic myogenic cells can be evaluated visually or by means of automated reading.

The principle of quantification by automated reading is based on finding the ratio of the area of desmin-positive cells to the number of Giemsa-stained nucleated cells.

The background noise is defined by the level of labelling without primary antibody, and is systematically subtracted from the rate of positive labelling.

Measurement of the ratio makes it possible to identify and count the proportion of labelled cells having the desired phenotype at the expected time.

This type of technology can be widely used for all cell types for which there are specific proteins and antibodies directed against the latter.

In another version of the method it is possible to count the total number of cells using ubiquitous markers. As an example non-muscle actin, vimentin, tubulin or transcription factors such as SP1 may be mentioned.

### Selection by Freezing and Preservation Using Trehalose

Selection of cells, and in particular of muscle cells, skeletal muscle stem cells, muscle stem cells, muscle precursor stem cells, myoblasts or satellite cells can also be achieved, both in continuous and in sequential extraction, by freezing the cells in a cryo-protective medium, selecting certain cells preferentially. The addition of trehalose at concentrations ranging from 1 mM to 0.005 M in the freezing medium makes possible the selective preservation of muscle cells with significant ability to regenerate. Other compounds that can allow such selection by freezing are the sugars in general, glycine, HES (hydroxyethyl starch), glycerol or arbutin.

Similarly, the addition of trehalose or of sugars in general, and in particular glycine, HES (hydroxyethyl starch), glycerol or arbutin to a medium for storage or preservation allows, with or without freezing, positive selection of muscle cells, in particular of skeletal muscle stem cells, muscle stem cells, muscle precursor stem cells, myoblasts or satellite cells.

### Improved Culture Media

The invention also proposes improved culture media and in particular selection media for myoblasts. It was discovered that, surprisingly, the media of the prior art could be formulated for the culture and selection of myoblasts and muscle stem cells without using insulin. The invention therefore proposes insulin-free culture and selection media. It was also discovered that, surprisingly, the ascorbic acid that was used by the media of the prior art could be replaced completely or partially with ascorbic acid 2-phosphate. Ascorbic acid 2-phosphate is the precursor of ascorbic acid. This formulation leads to greater stability and therefore greater bioavailability of ascorbic acid in vivo.

### Example 1

### Comparative Analysis of the Conditions of Extraction on Muscle Samples of Human Origin in the Production of Cells with Therapeutic Potential

This example involves comparing two extraction protocols, namely sequential extraction and continuous extraction, the subject of the present invention. Cell extraction is the second step of the cell production process. It makes it possible to release the cells from the tissue sample (biopsy) taken from the patient.

The experiments that were used for evaluating the two extraction methods were carried out on 5 samples of human muscle tissues obtained from different patients.

### Methods

Each muscle sample undergoes two parallel extraction methods: the sequential method (i.e. according to the prior art) and the continuous method according to the invention. This step of extraction is followed by steps of amplification and of freezing. The results of cell characterization obtained are presented below.

### Description of the Two Extraction Methods

The first step is common to both extraction methods. Once it has been removed from its transport medium, the biopsy is placed in a sterile culture dish with a few drops of DMEM/F12 so that it does not dry out. The adipose tissues and the aponeuroses are removed using a disposable scalpel, then the biopsy is cut up into small pieces with surgical scissors. This is the first step of mechanical dissociation, which is common to both methods.

### Sequential Extraction Method

A volume of 4 mL of collagenase NB6 at 0.5 mg/ml (enzyme solution I) is added to the pieces of comminuted tissues. Digestion is carried out at 37° C. for 10 minutes, under gentle stirring.

The suspension is then centrifuged at up to 500 rpm, and the cells contained in the supernatant are recovered in a 50-mL tube. A volume of 4 mL of DMEM/F12+20% fetal calf serum is added to the cells.

A volume of 4 mL of trypsin/EDTA (enzyme solution) is added to the tissue fragments. Digestion is carried out at 37° C. for 10 minutes.

The suspension is then centrifuged gently at up to 500 rpm, and the cells contained in the supernatant are collected in the 50-mL tube containing the cell suspension. A volume of 4 mL of DMEM/F12+20% fetal calf serum is added to the suspension.

The step of digestion with collagenase is carried out several times, as well as the step of digestion with trypsin (approximately 3 times for each step).

After each digestion step, a drop of suspension is deposited on a slide in order to observe the release of the sarcomeres under the microscope. The cell suspension is centrifuged at 1000 rpm for 5 minutes. The supernatant is removed and the pellet is taken up in DMEM/F12. Cell counting is difficult at this stage. Normalization is based on the weight of tissue.

Sequential digestion therefore comprises the following steps:
1. Tissue+enzyme solution I.
2. Slow centrifugation for separating the undigested tissues (pellet) and the cells released by the action of solution I (supernatant).
3. Addition of one volume of stopping medium to one volume of supernatant.
4. Undigested tissue+enzyme solution II (trypsin/EDTA).
5. Slow centrifugation for separating the undigested tissues (pellet) and the cells released by the action of solution II (supernatant).
6. Addition of one volume of stopping medium to one volume of supernatant.
7. The undigested tissue is ready to undergo a new digestion cycle.

This type of digestion requires two enzymes (collagenase and trypsin), a chelating agent for divalent ions (EDTA) and a considerable time for manipulation including numerous stages of cellular distribution and centrifugation making this stage difficult to carry out under GMP conditions. It should be added that the activity of an enzyme such as trypsin is sensitive to the presence of serum proteins, which can inhibit its activity. This stage is subject to variations and is therefore operator-dependent.

The compositions of the enzyme solutions are as follows:

### Enzyme Solution I

Blendzyme3 (collagenase) 25 μg/m/gentamicin, 50 μg/ml in DMEM/F12.

### Enzyme Solution II

Gentamicin 50 μg/ml in DMEM/F12, trypsin 0.5 g/l, EDTA 0.2 g/l

### Continuous Extraction Method

The principles of this continuous extraction method are extraction under conditions allowing cell viability and selection. Selection is based on the ability to survive under defined conditions of the media and on preferential adhesion to the substrate.

This method combines extraction and selection in the same step, at least partially. In this example, selection is based on two properties: the ability to respond to specific growth factors and the ability of selective adhesion to the culture plastic.

After undergoing the mechanical extraction that is common to both methods, from 10 to 100 mg of tissue is placed in 6 mL of selection medium (DMEM/F12+αMEM+10% FCS+dexamethasone+sodium selenite+ascorbic acid+ascorbic acid 2-phosphate) in the presence of collagenase (0.5 mg/mL) (in a 25-cm² bottle and in 6 ml of medium). This step can be carried out in media without protein of non-human origin. The culture bottle is placed in a culture incubator (37° and 5% CO₂). After digestion for 24 hours, the supernatant, containing the non-adherent cells, is recovered and centrifuged at 1000 rpm for 5 minutes. The pellet is taken up in the following expansion medium (DMEM/F12+αMEM+10% FCS+FGF+dexamethasone+ascorbic acid) and seeded in 75-cm² bottles for the amplification phase. In this stage two types of cells are separated: the adherent cells, which are capable of adhering to the culture plastic for the 24 hours of enzymatic treatment and secondly the non-adherent cells, which remain in suspension under these conditions. It is these last-mentioned cells that are used in the remainder of the test. The two types of cells are the source of cells for subsequent amplifications.

The adherent cells from the 25-cm² bottle are cultured in a growth medium, then amplified.

The combined digestion and selection medium used is described in the following table.

TABLE 1
Combined digestion and selection medium

| DMEM/F12: alphaMEM (1/1) | Final concentration |
|---|---|
| Collagenase NB6 | 0.5 mg/ml |
| Gentamicin | 50 μg/ml |
| Fetal calf serum | 10% |
| Dexamethasone | 5.10⁻⁹ M |
| Ascorbic acid | 0.252 mM |
| Ascorbic acid 2-phosphate | 1 mM |
| Sodium selenite | 250 nM |

For the remainder of the comparative test, the cells extracted by the sequential method and the continuous method are subjected to the same experimental conditions.

### Characterization of the Cells Obtained by the Two Extraction Methods

A comparative study of the results of the two extraction methods was undertaken for the cells obtained from five patients.

The following parameters were analyzed for each patient:
The number of cells produced per mg of tissue per day after the first amplification.
The number of days of culture required to obtain 200 million cells from 500 mg of tissue.
The cell doubling time during the amplification process.

The cells extracted by the sequential method and the continuous method are subjected to the same experimental conditions after their extraction. These cells are amplified for a period varying between 10 and 15 days and are then frozen. The amplification period makes it possible to study the quantitative effects of the different extraction methods on cell production.

Cell amplification: the purpose of this step is to obtain amplification of the muscle cells by restricting the culture time to the minimum. This production phase takes place in an open system.

Cell culture is carried out in 2 phases including 3 successive steps in 636 cm² ventilated trays (Cell Stack, Corning®) or 75 cm² TPP® bottles=1 selection step and 2 cell amplification steps.

The first amplification phase (75 cm² TPP® bottles or Cell Stack, Corning®) comprises the selection and the first expansion which lasts 8 to 9 days. The second amplification phase (TPP® bottles or Cell Stack® trays) comprises the second expansion which lasts 3 to 10 days.

During the initial seeding, the extracted cells are cultured in the presence of the selection medium. The cell quantity is expressed in mg of biopsy from which the cells are extracted. This type of quantification was adopted because counting of the cells obtained from enzymatic digestion is not possible, owing to the presence of much tissue debris and the absence of cell individualization.

Thus, for 1 gram of biopsy, the equivalent of 200 mg is seeded in four 75-cm² bottles with 20 mL of selection medium in each bottle and the equivalent of 400 mg is seeded in two 636 cm² trays with 150 mL of selection medium (i.e. 0.7 mg/cm²).

### First Expansion

The medium is changed after 2-6 days. Change of medium takes place twice weekly.

### Second Expansion

All of the cells are detached by the action of the harvesting medium.

The harvesting medium is in contact with the cells for 5 to 10 minutes, then the cell suspension is taken up in the stopping medium. The detachment of the cells is verified with the inverted microscope.

After counting, the cells are reseeded in the expansion medium at a rate of 5×10⁴ cells per bottle or 4.5×10⁵ cells per tray (i.e. 700 cells per cm²). This is the second amplification phase, which lasts 3-10 days. In parallel with the seeding of the trays or 75-cm² bottles, three 25-cm² bottles are also seeded at an equivalent cell density (17.5×10³ cells).

After this second phase, the cells are detached by the action of the harvesting medium and then diluted in the stopping medium, to which 1.6% human albumin is added. The new cell suspension is then washed by centrifugation (200 g for 5 minutes) and taken up in the stopping medium supplemented with albumin 1.6%. Two successive washings are carried out. The count and the cell viability are measured between the two washings.

The number of bottles in the first phase is adjusted according to the weight of the biopsy obtained, and the number of bottles required for the second phase depends on the effectiveness of the first expansion.

### Culture Media Used

The most suitable culture medium for growth of the progenitor cells is an equal-volume mixture of Dulbecco's Modified Eagle's Medium/Ham F 12 (DMEM/F12) and alpha Modified Eagle's Medium (αMEM).

### Selection Medium

This is composed of DMEM/F12 and αMEM in 1/1 ratio (v/v) containing 10% foetal calf serum and gentamicin, supplemented with the following products at concentrations expressed as final concentrations:
Dexamethasone: 5.10⁻⁹ M
Ascorbic acid 2-phosphate: 1 mM
Ascorbic acid: 0.252 mM
Sodium Selenite: 250 nM

### Expansion Medium

This is composed of DMEM/F12 and αMEM (v/v) containing 10% foetal calf serum and gentamicin, supplemented with the following products, in amounts expressed as final concentration:
FGF b: 10 ng/mL
Dexamethasone: 5.10⁻⁹ M
Ascorbic acid 2-phosphate: 1 mM
Ascorbic acid: 0.252 mM

### Harvesting Medium

### Trypsin 0.5 g/L and EDTA 0.2 g/L.

### Stopping Medium

DMEM/F12 alone.

### Cell Freezing

Freezing makes the method safe and simplifies the logistics. After the last centrifugation, the cells are resuspended in the stopping medium+albumin 1.6%, which will be supplemented with DMSO in order to obtain a defined freezing medium, at constant temperature (greater than or equal to 20° C. and less than 25° C.). The cells are stored in freezing ampoules and the ampoules are placed in gaseous nitrogen. The cellular material obtained from an individual patient is arranged in a batch of ampoules. This batch is made up of at least 3 freezing ampoules:

### Results

In order to study the effects of the two extraction methods (sequential and continuous) on the method for producing cells with therapeutic potential, we analyzed 3 parameters characterizing the cells produced.

For each patient, we analyzed the following parameters:
The number of cells produced per mg of tissue per day after the first amplification.
The number of days of culture required to obtain 200 million cells from 500 mg of tissue.
The cell doubling time during the amplification process.

The results obtained on five independent biopsy samples (N=5) by 3 different testers are presented in the following table:

TABLE 2
Comparison of cell growth depending on the extraction method

| Extraction method | sequential | continuous | T test |
|---|---|---|---|
| Number of cells/mg of | 1088 +/− 1351 | 4351 +/− 2000 | p = 0.0176 |
| tissue/day | | | |
| Number of days to | 17.5 +/− 1.9 | 11 +/− 1 | p = 0.0002 |
| obtain 200 million | | | |
| cells with 500 mg of | | | |
| tissue | | | |
| Doubling time, hours | 20 +/− 2.7 | 20 +/− 4 | p = 0.8609 |

We can conclude from these results that continuous extraction:
   is a more effective extraction method and is subject to smaller interindividual variations;
   considerably reduces the culture time for obtaining the number of cells necessary for conducting our clinical test;
   does not significantly alter a biological parameter: the cell doubling time.

Continuous extraction is a method that is simpler to implement, and it greatly reduces the number of human manipulations.

### Long-Term Growth of the Cells Extracted According to the Two Extraction Methods

The purpose is to determine the effects of the two extraction methods on long-term growth and on the capacities for senescence of the cells thus produced.

The cells from two patients, treated by the two extraction methods, were cultured for 2 months and underwent 12 series of cell passages. At each passage, the number of cumulative divisions was calculated (see FIG. 1).

The results obtained indicate that there is no difference in cell growth between the cells treated by the two extraction methods. Under both conditions, a number of cumulative divisions between 35 and 45 was observed. After 30 divisions, the dividing time gets longer and there is senescence of the cells extracted according to the two methods. In conclusion, the continuous type of extraction does not alter the long-term growth of the cells and their senescence ability. This last-mentioned biological property, which indicates the non-transformed character of the cells thus produced, is also a guarantee of safety of the cellular therapeutic product.

This continuous extraction method makes it possible to:
carry out enzymatic digestion under conditions allowing the viability and the biological properties of the cells to be maintained;
combine extraction and cell selection in a single step;
reduce the number of human manipulations;
reduce the variability of the production process;
reduce the culture time for production of the required number of cells;
produce cells having capacities for long-term growth while maintaining their senescence ability, which is a guarantee of therapeutic safety.

### Example 2

### Analysis of the Effects of Continuous Extraction on the Cell Specifications Required for Obtaining Cells with Therapeutic Potential for Conducting Therapeutic Tests for the Repair of Small Muscles Such as the Urethral or Anal Sphincter

After transport, the muscle tissue is cut into small pieces mechanically. Cell extraction then takes place by enzymatic digestion.

Extraction is of the continuous type in order to ensure an optimum contact time between the cells and the enzyme and under conditions allowing the biological properties of the cells to be maintained. The action of the enzyme is inhibited by washing: dilution in the selection medium and centrifugation.

### Method:

Once transported, the muscle tissue is checked in, weighed and then cut into small pieces mechanically using surgical scissors and sterilized disposable tweezers.

The biopsy is placed in a dish with a few drops of DMEM/F12 so that it does not dry out. The adipose tissue and the aponeuroses are excised using a disposable scalpel, then the biopsy is cut up into small pieces with surgical scissors.

### Step 1. Continuous Extraction/Selection

The comminuted tissue is placed in the enzymatic digestion medium containing collagenase NB6 at 0.5 mg/ml.

Then the tissue fragments are deposited on a 636-cm² tray at a rate from 0.4 g to 1.2 g per tray, containing 150 ml of digesting medium (i.e. from 0.6 to 1.8 mg/cm²). This step can be carried out on a series of supports, including ventilated 75-cm² bottles.

The muscle tissue then undergoes a single cycle of enzymatic digestion by collagenase NB6.

The duration of enzymatic treatment is 24 hours. The treatment temperature is 37° C. and the CO₂ concentration is 5%. After digestion, the supernatant containing the medium and cells released in the medium is recovered and then centrifuged (200 g for 5 minutes). The sedimentation pellet is taken up in selection medium, and the sediments are reseeded in culture supports at a rate of 0.7 mg/cm².

The combined digestion and selection media are described in the following table:

TABLE 3
Combined digestion/selection medium

| DMEM/F12: alphaMEM (1/1) | |
|---|---|
| Collagenase NB6 | 0.5 mg/ml |
| Gentamicin | 50 μg/ml |
| Fetal calf serum | 10% |
| Dexamethasone | 5.10⁻⁹ M |
| Ascorbic acid | 0.252 mM |
| Ascorbic acid 2-phosphate | 1 mM |
| Sodium selenite | 250 nM |

### Cell Freezing Step

The optional freezing step can ensure safety of the method and simplify the logistics. After the last centrifugation, the cells are resuspended in the stopping medium+1.6% albumin which will be supplemented with DMSO in order to obtain a defined freezing medium, at constant temperature (greater than or equal to 20° C. and less than 25° C.). The cells are stored in freezing ampoules and the ampoules are placed in gaseous nitrogen. The cellular material obtained from an individual patient is organised in an ampoule batch. This batch is made up of at least 3 freezing ampoules.

The entire freezing operation is carried out by automatic systems, which allow the temperature drop to be controlled at a rate comprised between 1° C. and 2° C. per minute.

For 15 minutes following preparation of the cells, the ampoules are kept in the tank of the automatic freezing apparatus.

Automatic freezing involves the use of a temperature-reducing programmer called “DIGITCOOL”. This programmer allows automated, progressive temperature reduction. The temperature reduction validated during the pilot test is that used for freezing haematopoietic stem cells.

The product is introduced when the tank of the apparatus is at +10° C., then the tank temperature is lowered to the intermediate level, at −40° C. Sensitive heating allows the temperature to return to −25° C. and the temperature is lowered again, to −120° C. The ampoules will then be transferred in nitrogen vapour. Temperature reduction takes 1 hour.

The stability studies described demonstrate that cells that have undergone a step of cryopreservation have biological properties at least as good as cells that have not undergone cryopreservation. This bioequivalence was demonstrated in two different animal studies. The main criterion adopted in vitro is the clonal efficiency, and that evaluated in vivo is the cells' ability to colonize the urethra of the female rat.

### The Freezing Medium Used is Composed of:

DMEM/F12 at 1/1 ratio (v/v).
Cryoprotective factor: DMSO 5%.
1.6% human albumin (16 g/L).

### Controls

The set of controls is described in the following table

TABLE 4
Quality control

| Production stage | Quality control |
|---|---|
| Comminution of the biopsy fragment | VIROLOGICAL STATUS AND |
| | WEIGHING |
| Cell extraction by enzymatic | MICROSCOPIC OBSERVATION |
| digestion | MICROBIOLOGICAL CONTROL |
| Detachment of the cells | COUNTING |
| Second cell amplification | IN-PROCESS CONTROL |
| | COUNTING-VIABILITY |
| | MICROBIOLOGICAL CONTROL |
| Freezing | CHARACTERIZATION of the |
| | batch. |

### Control on the First Amplified Cell Suspension (Counting)

At the end of the first amplification phase, the cells are harvested and monitored by cell counting. Counting for a sample is carried out by reading the number of cells obtained on a Malassez slide.

This control determines the cell seeding stage for the second expansion.

### Control on the Second Cell Suspension (Counting)

This control, called an in-process control, is carried out during the second amplification (3^{rd} or 4^{th} day and 6^{th} or 7^{th} day) on a 25-cm² bottle intended for this use. This key step is critical for determining the day of freezing of the cell batch. The target density is from 50 000 to 150 000 cells per cm², and this can be obtained in 3 to 10 days.

The combined digestion/selection media are described below.

TABLE 5
Composition of the combined digestion/selection media including
enzyme solution I

| | | | | | | Unit/quantity |
|---|---|---|---|---|---|---|
| | | | | | | or percentage |
| | | | | Registration | Reference to | or final |
| Constituent | Supplier | Origin | Function | number | standards | concentration |
| DMEM/F12 | Cambrex | NOA | Culture medium | BE12-719F | | ½ volume |
| alphaMEM | Cambrex | NOA | Culture medium | BE12-169F | | ½ volume |
| Gentamicin | Schering | | antibiotic | AMM 322 349 1 | European | 50 mg/L |
| | Plough | | | | Pharmacopoeia | |
| | | | | | current edition | |
| Collagenase | Serva | OA | Enzymatic | | | 500 mg/L |
| (NB6) | | | digestion I | | | |
| Fetal calf serum | Cambrex | OA + | Growth factors | US14-417F | | 10% |
| | | irradiated | | | | |
| Ascorbic acid | Roche | chemical | antioxidant | AMM 342135-7 | European | 0.252 mM |
| | | | | or | Pharmacopoeia | |
| | | | | AMM 557160-6 | current edition | |
| Ascorbic acid 2- | Cambrex | chemical | antioxidant | A8960 | | 1 mM |
| phosphate | | | | | | |
| Dexamethasone | Merck | chemical | growth cofactor | AMM 550977-7 | European | 5 10⁻⁹ M |
| | | | | or | Pharmacopoeia | |
| | | | | AMM 309740-2 | current edition | |
| Sodium selenite | Sigma | NOA | cofactor | S5261 | | 250 nM |

The composition of the freezing medium is described below.

TABLE 6
Composition of the freezing medium. The volume of DMSO added
corresponds to 5% of the volume of the final product.

| | | | | | | Unit/quantity |
|---|---|---|---|---|---|---|
| | | | | | | or percentage |
| | | | | Registration | Reference to | in the final |
| Constituent | Supplier | Origin | Function | number | standards | product |
| Albumin 20% | LFBx | human | Maintenance of | AMM 558 451-4 | European | 16 g/L |
| | | | oncotic pressure | | Pharmacopoeia | |
| | | | | | Current version | |
| DMSO | Braun | chemical | Cryoprotective | 9575 H | European | 5% |
| | | | agent | | Pharmacopoeia | |
| | | | | | Current version | |
| DMEM/F12 | Cambrex | chemical | Cellular | BE12-719F | European | q.s. 100% |
| | | | medium | | Pharmacopoeia | |
| | | | | | Current version | |

### Results

As continuous extraction makes it possible to obtain potentially therapeutic cells under better safety conditions, we analyzed the specifications of the cells produced using this technique on samples of muscle tissue from three patients.

The results are shown in the following table:

TABLE 7
Properties of the cells extracted by continuous extraction.

| Parameters | Expected results | Sample 1 | Sample 2 | Sample 3 |
|---|---|---|---|---|
| Viability | >70% | nd | 97% | 74% |
| Coverage | >50% | 98% | nd | 79.9 |
| Clonal | >10% | 64% | 32% | 35% |
| efficiency | | | | |
| Doubling time | Between 13.5 | 19.3 | 17 | 16 |
| (hours) | and 30 | | | |
| Expression of | >50 | 153 | 118 | 230 |
| desmin | | | | |

nd = not determined

For all the parameters analyzed, the cells produced using the extraction technique meet the specifications required for conducting our cell therapy test.

In conclusion, continuous extraction allows production of potentially therapeutic cells under improved safety conditions, having the cell specifications required for conducting therapeutic tests for the tissue repair of muscle by cell therapy.

### Example 3

### Influence of the Digestion/Selection Time on the Effectiveness of Cell Extraction for Cell Production

In this example, we analyzed the influence of the enzymatic digestion/selection time on the number of extracted cells that are capable of proliferation.

### Method

The extraction method is described in Example 2. The parameter studied is the number of cells extracted per mg of tissue per day after a first amplification.

### Results

We chose the number of cells obtained after 24 hours as reference, representing 100%. The data obtained for two patients are presented in the following table.

TABLE 8
Number of cells as a function of time after the first amplification

| Duration of combined | |
|---|---|
| digestion/selection | Number of cells/mg of tissue/day |
| 6 hours | 69% |
| 24 hours | 100% |
| 30 hours | 407% |

The number of cells thus obtained depends on the duration of digestion/selection. Passing from 24 hours to 30 hours makes it possible to increase the effectiveness of extraction by a factor of 4. As the average cell division time is of the order of 24 hours, the culture time for producing the required number of cells for a therapeutic test can be reduced by 2 days. The duration of extraction/selection is an important step of the process and its efficiency depends on the duration of digestion/selection.

### Example 4

### Cell Selection by Differential Adhesion

In this example we shall analyse the cells selected and the cells not selected by the so-called continuous extraction method.

### Methods

The samples originate from surgical waste obtained in the course of surgical operations. The samples are obtained under sterile conditions and are transported in a medium that maintains tissue viability. On arrival in the laboratory, the tissues undergo various operations. Cellular tissues are always composed of numerous cell types, and one of the aims of this method is to increase the homogeneity of the cells produced. There are numerous strategies for achieving these aims. Cell selection can be based on biological properties (survival ability, selective cytotoxicity, gene expression, adhesion ability), or on molecular properties (presence of membrane markers). The selection techniques can be either culture techniques or techniques employing magnetic sorting or flow cytometry.

For this study, the properties of differential adhesion to treated plastic for cell culture were used. The steps of digestion and selection were carried out according to Example 1.

During the step of combined enzymatic digestion/selection, the tissue fragments are placed in dishes of plastic treated for cell culture. With this method it is therefore possible to separate the cells that will adhere to the substrate (adherent cells) and the cells that will not adhere to the substrate (non-adherent cells). After culture for 24 hours the non-adherent cells were separated from the adherent cells.

The non-adherent cells are recovered using a pipette. In this step the medium contains the growth factors and the cells released by enzymatic digestion. This cell suspension of non-adherent cells thus obtained is a source of cells for cell preservation, selection and cell amplification as for Example 1.

The adherent cells are the cells that remain attached to the substrates after pipetting. The cells are then amplified directly or subcultured using trypsin EDTA solution. These cells are a source of cells for cell preservation, selection and cell amplification. The conditions of growth are similar to those used for the adherent cells.

### Results

The non-adherent cells and the adherent cells are cultured. Their characteristics are shown in the following table. The techniques used are described in Example 1.

TABLE 9
Characteristics of the cells in relation to adherence

| | Non-adherent cells | Adherent cells |
|---|---|---|
| Number of cells/mg of tissue/day | 3222 | 596 |
| after the 1st amplification | | |
| Doubling time, hours | 20.7 | 28.2 |
| Expression of desmin (c) | high | not detectable |

The selection method using preferential adhesion to plastic treated for cell culture makes it possible to separate two cell populations. The two populations exhibit growth ability in culture. The non-adherent cells are 5 times less numerous than the adherent cells and their doubling time is nearly 30% lower. Furthermore, they are very different with respect to expression of desmin. Almost all the non-adherent cells express desmin, whereas the situation is reversed for the adherent cells. To summarize, this method of selection makes it possible to separate muscle cells (non-adherent cells) from non-muscle cells (adherent cells), obtained from a muscle biopsy. This method is simple to implement and can be carried out under GMP conditions, the conditions necessary for the production of therapeutic cells.

### Example 5

### Cell Digestion/Selection on Frozen Tissue Fragments

The possibility of freezing the tissue fragments can simplify the logistics of the cell production methods.

The samples originate from surgical waste obtained in the course of surgical operations. The samples are obtained under sterile conditions and are transported in a medium that maintains tissue viability. On arrival in the laboratory, the tissues undergo various operations. The tissue fragments were frozen directly after the step of mechanical dissociation. Freezing can be carried out in the presence of serum components or of defined media. The samples thus obtained are stored in liquid nitrogen vapour.

Freezing can ensure safety of the method and can simplify the logistics. After the last centrifugation, the cells are resuspended in the stopping medium+albumin 1.6%, which will be supplemented with DMSO in order to obtain a defined freezing medium: without proteins of animal origin, at constant temperature (greater than or equal to 20° C. and less than 25° C.). The cells are stored in freezing ampoules and the ampoules are placed in gaseous nitrogen. The cellular material obtained from an individual patient is organised in an ampoule batch. Thawing is rapid, and is carried out between 35° C. and 37° C.

### Methods

Once they are taken out of the nitrogen container, the tissue fragments are thawed rapidly at a temperature of 37°.

These fragments undergo digestion/selection of the continuous type as in Examples 1 and 2.

### Results

The following table compares the results obtained with fresh or frozen fragments obtained from an identical sample of human tissue. The fragments were frozen in various freezing media. The concentration of DMSO is identical (5% of the final volume) under all conditions. Certain of these conditions are free from all proteins of animal origin.

TABLE 10
Percentage of cells preserved in relation to the freezing media

| | Number of cells | |
|---|---|---|
| | obtained/day/mg | |
| | of tissue | Percentage |
| Fresh tissue without steps | 3322 cells/mg/day | 100% |
| of previous freezing | | |
| DMEM/F12 + albumin + | 2240 cells/mg/day | 67% |
| 5% DMSO | | |
| DMEM/F12: 95% FCS + | 1440 cells/mg/day | 43% |
| 5% DMSO | | |

It is important to note that in a medium totally free from proteins of animal origin, it is possible to preserve 67% of the cells capable of being amplified and therefore able to supply therapeutic cells. The presence of calf serum in the preservation medium lessens the effectiveness of the freezing medium.

In the second part of the study we were interested in the qualitative and quantitative criteria of the cells thus obtained after freezing the tissue. In all cases it is possible to observe desmin-positive cells.

TABLE 11
Comparison of the effect of freezing of tissues

| | DMEM/F12 + | DMEM/F12: |
|---|---|---|
| | albumin + 5% | 95% FCS + |
| Condition | DMSO | 5% DMSO |
| Number of tissues frozen | 100 | 100 |
| Number of days of 1st amplification | 10 | 10 |
| Number of cells after the 1st | 2 240 000 | 1 440 000 |
| amplification | | |
| Number of cells/mg of tissue/day after | 2240 | 1440 |
| the 1st amplification | | |
| Number of cells seeded for the 2nd | 200 000 | 200 000 |
| amplification | | |
| Number of days of 2nd amplification | 7 | 7 |
| Number of cells produced after the 2nd | 57 040 000 | 49 040 000 |
| amplification | | |
| Doubling time (hours) | 20.6 | 21.2 |
| Number of cells/cm² after the 2nd | 190 133 | 163 467 |
| amplification | | |
| Number of days to obtain 200 million | 13.6 | 14.2 |
| cells with 500 mg of tissue | | |

In the absence of proteins of non-human origin (DMEM/F12+albumin+5% DMSO), it is possible to preserve tissues by selecting the muscle cells. Under these conditions, it is possible to obtain a better selection yield (compared to that observed in the presence of fetal calf serum) in digestion/selection and to increase the ability to select muscle cells. Under these conditions, the cells are capable of self-renewal and of expressing desmin and forming the precursors of muscle fibres: myotubes. Under these conditions, in the absence of proteins of animal origin, 16 days would suffice to accumulate 200 million potentially therapeutic cells.

Direct freezing of muscle tissue in the absence of proteins of animal rigin makes it possible to store cells having therapeutic potential in conditions compatible with good laboratory practice necessary for the production of therapeutic cells.

### Example 6

### Cell Preservation by Freezing after the Step of Combined Digestion and Selection

In this example, we tested the possibility of preserving the cells by freezing, by freezing after the first step of digestion and selection. The possibility of preserving the cells at several steps of the production process is important for technical reasons of a regulatory and administrative nature.

### Methods

As in the previous examples, the samples originate from surgical waste obtained in the course of surgical operations. The samples are obtained under sterile conditions and are transported in a medium that maintains tissue viability.

The biopsy is placed in a dish with a few drops of DMEM/F12 so that it does not dry out. The adipose tissue and the aponeuroses are excised using a disposable scalpel, then the biopsy is cut up into small pieces with surgical scissors.

### Step 1. Continuous Digestion/Selection:

The comminuted tissue is placed in the medium for enzymatic digestion containing collagenase NB6 at 0.5 mg/ml.

Then the tissue fragments are deposited on a 636-cm² tray at a rate from 0.4 g to 1.2 g per tray containing 150 ml of digesting medium (i.e. from 0.6 to 1.8 mg/cm²). The muscle tissue then undergoes a single cycle of enzymatic digestion by collagenase NB6.

The duration of enzymatic treatment is 24 hours. The treatment temperature is 37° C.

After digestion, the supernatant containing the medium+cells released in the medium is recovered and then centrifuged (200 g for 5 minutes).
The combined digestion/selection media are composed of:

| DMEM/F12: alphaMEM (1/1) | Final concentration |
|---|---|
| Collagenase NB6 | 0.5 mg/ml |
| Gentamicin | 50 μg/ml |
| Fetal calf serum | 10% |
| Dexamethasone | 5.10⁻⁹M |
| Ascorbic acid | 0.252 mM |
| Ascorbic acid 2-phosphate | 1 mM |
| Sodium selenite | 250 nM |

After a washing step, the cellular supernatant containing the non-adherent cells is frozen in the growth medium described below.

### Freezing Medium:

### DMEM/F12

Cryoprotective factor: DMSO 5%
1.6% human albumin (16 g/L)

### Results

The tissue is subjected to a procedure of combined digestion and selection according to Example 1. After 24 hours of extraction/selection, the cells contained in the supernatant are either cultured directly as in Example 1, or frozen under different experimental conditions.

After thawing, the cells' capacities for growth are analyzed and compared with the results obtained with cells that did not undergo freezing. The results are shown in the following table:

TABLE 12
Percentage of cells preserved in relation to the freezing media

| | Number of cells | |
|---|---|---|
| | obtained/day/mg of tissue | Percentage |
| No freezing | 7291 | 100% |
| DME/F12, albumin, DMSO 5% | 2857 | 39% |
| DME/F12, albumin, FCS 10%, | 2343 | 32% |
| DMSO 5% | | |
| DME/F12, albumin, FCS 10%, | 2514 | 34% |
| DMSO 10% | | |
| DME/F12, FCS 95%, DMSO 5% | 4486 | 61% |

After combined digestion and selection, the cells frozen directly can be used as a source of cells for the production of therapeutic cells. However, the effectiveness of the method under these conditions is partial and depends on the conditions of freezing. From a qualitative standpoint, freezing does not alter the properties of the cells preserved in this way. Cells extracted from frozen tissue possess characteristics identical to those of cells extracted from fresh tissue, whether for growth properties or differentiation properties.

Preservation of cells by freezing after digestion and selection does not alter the potential of the cells. These cells exhibit both a self-renewal ability and a differentiation ability at least equal to the cells extracted from fresh tissue. This step of the method makes it possible to preserve the cells starting from the step of extraction/selection without altering the potential of the preserved cells. The cells preserved in this way can be sources of cells for cell therapy and for constitution of a tissue bank.

### Example 7

### Trehalose—an Agent for the Selective Preservation of Muscle Cells in a Tissue Frozen Immediately

The methods used for this example are similar to the methods used for Example 5. In the following table, we were interested in the quantitative aspects of the cells extracted from tissue frozen directly. In this example the concentration of trehalose is 0.2M. The concentration can be varied from 1 mM to 1M. The results are shown in the following table:

TABLE 13
Comparison between the number of cells obtained after freezing
with or without trehalose

| | Number of cells | |
|---|---|---|
| | obtained/day/mg of tissue | Percentage |
| Fresh tissue without previous | 3322 cells/mg/day | 100% |
| freezing steps | | |
| DMEM/F12 + albumin + 5% | 2240 cells/mg/day | 67% |
| DMSO | | |
| DMEM/F12 + albumin + 5% | 600 cells/mg/day | 18% |
| DMSO + 0.2 M trehalose | | |

In the presence of trehalose, cells can be extracted from the tissues directly, though in smaller number. On the one hand, this effect is due to the high concentration of trehalose (0.2M) used under this experimental condition. The results in the following table relate to the capacities for growth of cells extracted from frozen tissues in the presence and in the absence of trehalose.

TABLE 14
Comparison of the effect of freezing of tissues in the presence of
trehalose on extraction and cell amplification

| | | DMEM/F12 + |
|---|---|---|
| | DMEM/F12 + | albumin + 5% |
| | albumin + 5% | DMSO + 0.2M |
| Condition | DMSO | trehalose |
| Number of tissues frozen | 100 | 100 |
| Number of days of 1st | 10 | 10 |
| amplification | | |
| Number of cells after the 1st | 2 240 000 | 600 000 |
| amplification | | |
| Number of cells/mg of | 2240 | 600 |
| tissue/day after the 1st | | |
| amplification | | |
| Number of cells seeded for the | 200 000 | 200 000 |
| 2nd amplification | | |
| Number of days of 2nd | 7 | 7 |
| amplification | | |
| Number of cells produced after | 57 040 000 | 29 040 000 |
| the 2nd amplification | | |
| Doubling time (hours) | 20.6 | 23.4 |
| Number of cells/cm² after the | 190 133 | 96 800 |
| 2nd amplification | | |
| Number of days to obtain 200 | 13.6 | 15.9 |
| million cells with 500 mg of | | |
| tissue | | |

The capacities for growth of the cells extracted in the presence of trehalose are very close to the cells extracted from unfrozen tissues. In fact, 16 days suffice to accumulate 200 million cells from 500 mg of tissue frozen in the presence of trehalose. Absence of this sugar only reduces the time by 2 days. Freezing in the presence of trehalose does not alter the growth abilities of the extracted cells. The clonal efficiency is the ratio of the number of colonies observed to the initial number of cells seeded. The following table shows that freezing in the presence of trehalose does not alter the clonal growth ability.

TABLE 15
Comparison of the clonal efficiency of cells frozen with or without
trehalose

| | Without trehalose | | | | | | With trehalose | | | | | |
|---|---|---|---|---|---|---|---|---|---|---|---|---|
| | Number of days of culture after thawing | | | | | | | | | | | |
| | 13 | | | 21 | | | 13 | | | 21 | | |
| Number of | 19 | 24 | 27 | 27 | 13 | 17 | 22 | 24 | 24 | 23 | 20 | 15 |
| colonies | | | | | | | | | | | | |

| | | | | |
|---|---|---|---|---|
| Clonal | 23.3 | 19 | 23.3 | 19.3 |
| efficiency (%) | | | | |

In the following table, we analyzed the ability of the cells extracted after freezing to express the desmin marker specific to muscle cells. The concentration of trehalose was 0.2M. After preservation in the presence or absence of trehalose, the tissues underwent a step of digestion and selection. The cells were analyzed for expression of desmin. The cells were labelled with immunoperoxidase by means of a specific antibody that recognizes desmin, a protein specific to muscle cells.

The vast majority of the cells extracted and selected from tissue frozen in the presence of trehalose express desmin and a greater proportion of them form myotubes, the precursor cells of muscle fibre. FIG. 2 shows very clearly that the vast majority of the cells extracted from tissue frozen in the presence of trehalose express desmin. This is confirmed by the results obtained by image analysis, presented in the following table.

TABLE 16
Comparison of the number of cells expressing desmin according
to whether freezing is carried out with or without trehalose

| Tissue frozen: | without trehalose | with trehalose |
|---|---|---|
| Desmin | 522 +/− 102 | 1544 +/− 286 |

It is very remarkable to find that after freezing in the presence of trehalose, the vast majority of the cells are capable of expressing desmin. In this sense, this is an agent that improves the selection of muscle cells with therapeutic potential during the tissue preservation step.

Freezing in a medium containing trehalose therefore makes it possible to preserve and to select muscle precursor cells, myoblasts or satellite cells so as to obtain an enriched population with the potential for cell regeneration and therefore repair.

Preservation of the tissue fragments by freezing does not alter the potential of the extracted cells. The presence of trehalose makes possible the selective preservation of the cells present in the tissue that will give cells capable of self-replication and of producing cells that express desmin. These cells have both self-renewal and differentiation abilities at least equal to the cells extracted from fresh tissues. This step of the method makes it possible to store tissue fragments without altering the potential of the cells present in said fragments. The fragments stored in this way can be sources of cells for cell therapy and for the constitution of a tissue bank.

The constitution of banks of tissues preserving the viability of the cells constituting the tissues is an intellectual and an industrial objective. The method described makes it possible to preserve the tissue organization and the selective cell viability of muscle cells. With the present invention, it is possible to construct banks of cellular tissue without steps of enzymatic extraction and without steps of cell culture.

1-44. (canceled)

45. A method of cell selection in a culture of cells, the method comprising at least one at least partially combined step of enzymatic digestion and selection in culture.

46. A method according to claim 45, wherein the source of the cells is a tissue sample.

47. A method according to claim 46, wherein the source of cells is a muscle biopsy.

48. A method according to claim 45, wherein the source of the cells is a cell culture.

49. A method according to claim 48, wherein the source of the cells is an aggregate of cells in culture.

50. A method according to claim 45, wherein the cells selected are muscle cells.

51. A method according to claim 50, wherein the cells selected are skeletal muscle stem cells, muscle stem cells, muscle precursor stem cells, myoblasts or satellite cells.

52. A method according to claim 45, wherein selection is carried out at least by culture in a selection medium.

53. A method according to claim 52, wherein the selection medium includes an enzyme used for enzymatic digestion.

54. A method according to claim 53, wherein enzymatic digestion uses an enzyme selected from a group comprising collagenase, neutral protease, pronase, trypsin and mixtures thereof.

55. A method according to claim 54, wherein the combined step of enzymatic digestion and selection takes at least 3 hours.

56. A method according to claim 54, wherein the combined step of enzymatic digestion and selection takes at least 12 hours.

57. A method according to claim 54, wherein the combined step of enzymatic digestion and selection takes at least 48 hours.

58. A method according to claim 45, wherein selection is carried out at least by adhesion to a culture substrate.

59. A method according to claim 58, wherein the substrate is chosen from a group comprising glass, treated plastic, synthetic substrates, nutrient cells, antibodies, peptides and constituents of an extracellular matrix.

60. A method according to claim 59, wherein the extracellular matrix comprises at least one of laminin, fibronectin, vitronectin.

61. A method according to claim 45, wherein selection is carried out at least by adhesion to a substrate and by culture in a selection medium.

62. A method according to claim 61, wherein adherent cells are selected.

63. A method according to claim 61, wherein non-adherent cells are selected.

64. A method according to claim 45, wherein selection is carried out by culture in a selection medium comprising at least dexamethasone, selenium, and one or more compounds chosen from a group comprising ascorbic acid 2-phosphate, ascorbic acid and mixtures thereof.

65. A method according to claim 64, wherein the culture medium includes an enzyme used for enzymatic digestion.

66. A method according to claim 65, wherein enzymatic digestion uses an enzyme selected from a group comprising collagenase, neutral protease, pronase, trypsin and mixtures thereof.

67. A method according to claim 66, wherein the combined step of enzymatic digestion and selection takes at least 3 hours.

68. A method according to claim 66, wherein the combined step of enzymatic digestion and selection takes at least 12 hours.

69. A method according to claim 66, wherein the combined step of enzymatic digestion and selection takes at least 48 hours.

70. A method for selecting muscle cells by a freezing step in the presence of a compound chosen from a group comprising sugars, trehalose, glycine, HES (hydroxyethyl starch), glycerol and arbutin.

71. A method according to claim 70, wherein the source of the cells is a tissue sample.

72. A method according to claim 71, wherein the source of the cells is a muscle biopsy.

73. A method according to claim 70, wherein the source of the cells is a cell culture.

74. A method according to claim 73, wherein the source of the cells is an aggregate of cells in culture.

75. A method according to claim 70, wherein the cells selected are skeletal muscle stem cells, muscle stem cells, muscle precursor stem cells, myoblasts or satellite cells.

76. A method according to the claim 75, wherein freezing is carried out in the presence of trehalose.

77. A method according to the claim 76, wherein a concentration of trehalose is between 1 mM and 1 M.

78. A method according to the claim 77, wherein the concentration of trehalose is 0.2 M.

79. Cells obtainable by the method of claim 45.

80. Muscle cells obtainable by the method of claim 45.

81. A method of therapy comprising administrating to a patient in need thereof, a therapeutic amount of muscle cells obtainable by the method of claim 45.

82. A method of therapy according to claim 81, wherein the therapy comprises a functional treatment of muscle.

83. A method of therapy according to claim 82, wherein the therapy comprises a functional treatment of small muscles.

84. A method of therapy according to claim 82, wherein the therapy comprises a functional treatment of sphincters.

85. A method of therapy according to claim 81, wherein the therapy comprises a functional treatment of urinary incontinence.

86. A method of therapy according to claim 81, wherein the therapy comprises a functional treatment of anal incontinence.

87. Cells obtainable by the methods of claim 70.

88. Muscle cells obtainable by the methods of the claim 70.

89. A method of therapy comprising administrating to a patient in need thereof, a therapeutic amount of muscle cells obtainable by the method of claim 70.

90. A method of therapy according to claim 89, wherein the therapy comprises a functional treatment of muscles.

91. A method of therapy according to claim 90, wherein the therapy comprises a functional treatment of small muscles.

92. A method of therapy according to claim 90, wherein the therapy comprises a functional treatment of sphincters.

93. A method of therapy according to claim 89, wherein the therapy comprises a functional treatment of urinary incontinence.

94. A method of therapy according to claim 89, wherein the therapy comprises a functional treatment of anal incontinence.

95. A culture medium that is substantially free of insulin, comprising at least dexamethasone, selenium, and one or more compounds chosen from a group comprising ascorbic acid 2-phosphate, ascorbic acid and mixtures thereof.

96. A culture medium according to claim 95 additionally comprising serum.

97. A culture medium according to claim 96, wherein the serum comprises fetal calf serum.

98. A culture medium according to claim 96, wherein the serum comprises human serum.

99. A culture medium according to claim 96, wherein the serum concentration by volume is at least 5%.

100. A culture medium according to claim 99, wherein the serum concentration by volume is at least 10%.

101. A culture medium according to claim 95 additionally comprising an enzyme for enzymatic digestion.

102. A culture medium comprising at least dexamethasone, selenium, ascorbic acid 2-phosphate and ascorbic acid.

103. A culture medium according to claim 102 additionally comprising serum.

104. A culture medium according to claim 103, wherein the serum comprises fetal calf serum.

105. A culture medium according to claim 103, wherein the serum comprises human serum.

106. A culture medium according to the claim 103, wherein the serum concentration by volume is at least 5%.

107. A culture medium according to the claim 106, wherein the serum concentration by volume is at least 10%.

108. A culture medium comprising at least dexamethasone at a concentration of approximately 5×10⁻⁹ M, ascorbic acid 2-phosphate at a concentration of approximately 1 mM, ascorbic acid at a concentration of 0.252 mM and sodium selenite at a concentration of 250 nM.

109. A culture medium according to claim 108 additionally comprising an enzyme for enzymatic digestion.

110. A culture medium according to the claim 109, wherein the enzyme is chosen from a group comprising collagenase, neutral protease, pronase, and trypsin.

111. A cell preservation medium comprising albumin, trehalose and DMSO.

112. A cell preservation medium according to claim 111, wherein a final concentration of trehalose is between 1 mM and 1 M.

113. A cell preservation medium according to claim 111, wherein a final concentration of trehalose is 0.2 M.

114. A cell preservation medium according to claim 112 comprising from 1 to 50% albumin by volume, from 0 to 20% DMSO by volume and from 1 mM to 1 M of trehalose.

115. A cell preservation medium according claim 114 additionally comprising serum.
